(19) United States
(12) Patent Application Publication
(10) Pub. No.: US 2018/0214126 A1
(43) Pub. Date: Aug. 2, 2018

(54) CAPACITIVE MICROMACHINED ULTRASONIC TRANSDUCER, PROBE AND METHOD OF MANUFACTURING THE SAME

(71) Applicant: Samsung Electronics Co., Ltd., Suwon-si, Gyeonggi-do (KR)

(72) Inventors: Young Il KIM, Suwon-si (KR); Kyung Il CHO, Seoul (KR); Bae Hyung KIM, Yongin-si (KR); Jong Keun SONG, Yongin-si (KR); Eun Sung LEE, Hwaseong-si (KR); Min Seog CHOI, Seoul (KR)

(21) Appl. No.: 15/745,100

(22) PCT Filed: Jul. 15, 2015
(86) PCT No.: PCT/KR2015/007368

§ 371 Date: Jan. 15, 2018

(51) Int. Cl. A61B 8/00; B06B 1/02; G01N 29/24

(52) U.S. Cl. CPC A61B 8/4488; A61B 8/4494; B06B 1/0207; G01N 29/2406; A61B 8/4405; B06B 1/0292

(57) ABSTRACT

Disclosed is a Capacitive Micromachined Ultrasonic Transducer (cMUT) including: an anchor; and a membrane coupled with the anchor, wherein the anchor includes at least one anchor groove. According to the cMUT, a probe, and a method of manufacturing the probe, it is possible to widen a contact area between the cMUT and a lens. Accordingly, it is possible to increase the interfacial strength between the cMUT and the lens while protecting the membrane of the cMUT. Also, the peeling phenomenon of the lens can be prevented, and the durability of transducers can be improved.

### CROSS-REFERENCE TO RELATED APPLICATION AND CLAIM OF PRIORITY

The present application claims priority under 35 U.S.C. § 365 and is a 371 National Stage of International Application No. PCT/KR2015/007368 filed Jul. 15, 2015, the disclosures of which are fully incorporated herein by reference into the present disclosure as if fully set forth herein.

### TECHNICAL FIELD

The present invention relates to a Capacitive Micromachined Ultrasonic Transducer (cMUT), a probe using the cMUT, and a method of manufacturing the probe.

### BACKGROUND

An ultrasound imaging apparatus irradiates ultrasonic waves to an object from the surface of the object, and receives ultrasonic waves (that is, echo ultrasonic waves) reflected from the object so as to acquire images about a target area inside the object, such as the sections of soft tissues or the flow of blood, based on the echo ultrasonic waves, thereby providing information about the target area.

The ultrasound imaging apparatus has advantages that it is a compact, low-priced apparatus and it has noninvasive and nondestructive characteristics, compared to other imaging apparatuses, such as an X-ray apparatus, a Computerized Tomography (CT) scanner, a Magnetic Resonance Image (MRI) apparatus, and a nuclear medical diagnostic apparatus. For the advantages, the ultrasound imaging apparatus is widely used to diagnose the heart, abdomen, urinary organs, uterus, etc.

The ultrasound imaging apparatus uses a probe which is ultrasonic waves generating means to acquire ultrasound images of an object. The probe includes at least one transducer to transmit ultrasonic waves to the object and to receive echo ultrasonic waves from the object. The ultrasound imaging apparatus acquires an ultrasound image of the object based on the echo ultrasonic waves.

Meanwhile, in response to a need for high-efficient transmission/reception of ultrasonic waves, a Capacitive Micromachined Ultrasonic Transducer (cMUT) which is a new concept non-contact ultrasonic transducer is being developed.

### SUMMARY

An aspect of the present disclosure is to provide a Capacitive Micromachined Ultrasonic Transducer (cMUT), a probe using the cMUT, and a method of manufacturing the probe.

In accordance with an aspect of the present disclosure, there is provided a Capacitive Micromachined Ultrasonic Transducer (cMUT) including: an anchor; and a membrane coupled with the anchor, wherein the anchor includes at least one anchor groove.

The membrane may include at least one membrane groove.

The anchor may be formed with at least one of Si, SiO₂, and SiNₓ.

The membrane may be formed with at least one of Al and Au.

An opened side of the anchor groove or an opened side of the membrane groove may have a polygon pattern or a circular pattern.

An opened side of the anchor groove and an opened side of the membrane may have different shapes of patterns.

An opened side of the anchor groove and an opened side of the membrane may have different sizes of patterns.

Sidewalls of the anchor groove or sidewalls of the membrane groove may be curved.

Sidewalls of the anchor groove or sidewalls of the membrane groove may be inclined.

The anchor groove and the membrane groove may be formed at different depths.

The anchor groove or the membrane groove may be formed using at least one of etching, deposition, and bonding.

In accordance with another aspect of the present disclosure, there is provided a probe including: a Capacitive Micromachined Ultrasonic Transducer (cMUT) comprising an anchor, and a membrane coupled with the anchor; and a lens coupled with the cMUT, wherein the anchor includes a least one anchor groove.

The membrane may include at least one membrane groove.

An opened side of the anchor groove or an opened side of the membrane groove may have a polygon pattern or a circular pattern.

An opened side of the anchor groove and an opened side of the membrane may have different shapes of patterns.

An opened side of the anchor groove and an opened side of the membrane may have different sizes of patterns.

Sidewalls of the anchor groove or sidewalls of the membrane groove may be curved.

Sidewalls of the anchor groove or sidewalls of the membrane groove may be inclined.

The anchor groove and the membrane groove may be formed at different depths.

In accordance with still another aspect of the present disclosure, there is provided a method of manufacturing a probe including: forming at least one anchor groove in an anchor; and applying a lens on the anchor and a membrane coupled with the anchor.

The method may further include forming at least one membrane groove in the membrane.

The forming of the anchor groove or the forming of the membrane groove may include forming the anchor groove or the membrane groove such that an opened side of the anchor groove or an opened side of the membrane groove has a polygon pattern or a circular pattern.

The forming of the anchor groove and the forming of the membrane groove may include forming the anchor groove and the membrane groove such that an opened side of the anchor groove and an opened side of the membrane have different shapes of patterns.

The forming of the anchor groove and the forming of the membrane groove may include forming the anchor groove and the membrane groove such that an opened side of the anchor groove and an opened side of the membrane have different sizes of patterns.

The forming of the anchor groove or the forming of the membrane groove may include forming the anchor groove or the membrane groove such that sidewalls of the anchor groove or sidewalls of the membrane groove are curved.

The forming of the anchor groove or the forming of the membrane groove may include forming the anchor groove or the membrane groove such that sidewalls of the anchor groove or sidewalls of the membrane groove are inclined.

The forming of the anchor groove and the forming of the membrane groove may include forming the anchor groove and the membrane groove at different depths.

The forming of the anchor groove or the forming of the membrane groove may include forming the anchor groove or the membrane groove using at least one of etching, deposition, and bonding.

According to the cMUT, the probe, and the method of manufacturing the probe, it is possible to widen a contact area between the cMUT and a lens. Accordingly, it is possible to increase the interfacial strength between the cMUT and the lens while protecting the membrane of the cMUT. Also, the peeling phenomenon of the lens can be prevented, and the durability of transducers can be improved.

### BRIEF DESCRIPTION OF THE DRAWINGS

FIG. 1 is a perspective view of an ultrasound imaging apparatus according to an embodiment of the present disclosure;

FIG. 2A shows a section of a probe including a transducer module;

FIG. 2B is an exploded perspective view of a transducer module;

FIG. 2C is a cross-sectional view showing Capacitive Micromachined Ultrasonic Transducers (cMUTs) mounted on an integrated circuit;

FIG. 3 is a view for describing a configuration of a cMUT;

FIG. 4 is a plan view of an element shown in FIG. 3;

FIG. 5A is a cross-sectional view showing a structure of a cell according to an embodiment of the present disclosure;

FIG. 5B is a cross-sectional view showing a structure of a cell according to another embodiment of the present disclosure;

FIG. 6A is a plan view of a cell according to an embodiment of the present disclosure;

FIG. 6B is a plan view of a cell according to another embodiment of the present disclosure;

FIGS. 7A to 7C are views for describing an example of an etching process;

FIG. 8 is a cross-sectional view showing an example of an anchor etched by an etchant;

FIG. 9 is a cross-sectional view showing another example of an anchor etched by an etchant;

FIGS. 10A to 10D are views for describing an etching process according to Deep Reactive-Ion Etching (DRIE);

FIGS. 11A to 11C are cross-sectional views showing examples of anchors on which deposited films are formed;

FIG. 12 is a flowchart illustrating a method of manufacturing a probe, according to an embodiment of the present disclosure; and

FIG. 13 is a flowchart illustrating a method of manufacturing a probe, according to another embodiment of the present disclosure.

### DETAILED DESCRIPTION

Hereinafter, a Capacitive Micromachined Ultrasonic Transducer (cMUT), a transducer module, and a method of manufacturing the transducer module, according to embodiments of the present disclosure, will be described in detail with reference to the accompanying drawings.

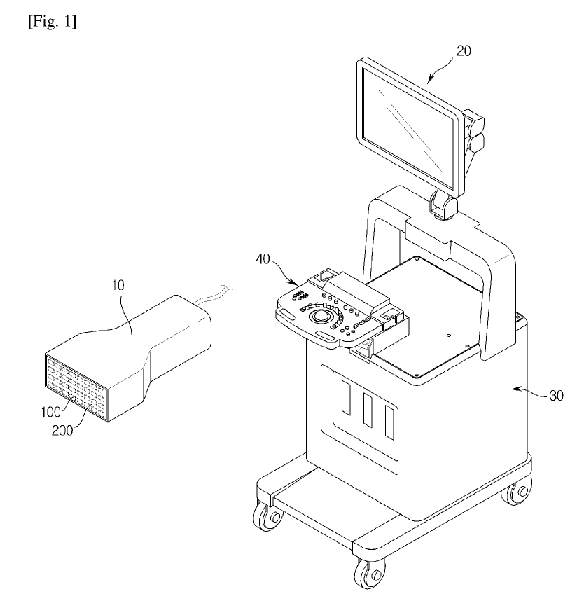

FIG. 1 is a perspective view of an ultrasound imaging apparatus according to an embodiment of the present disclosure.

Referring to FIG. 1, the ultrasound imaging apparatus may include a probe 10, a main body 30, an input unit 40, and a display unit 20.

The probe 10 may be connected to one end of a cable. The other end of the cable may be connected to a male connector (not shown). The male connector connected to the other end of the cable may be physically coupled with a female connector of the main body 30.

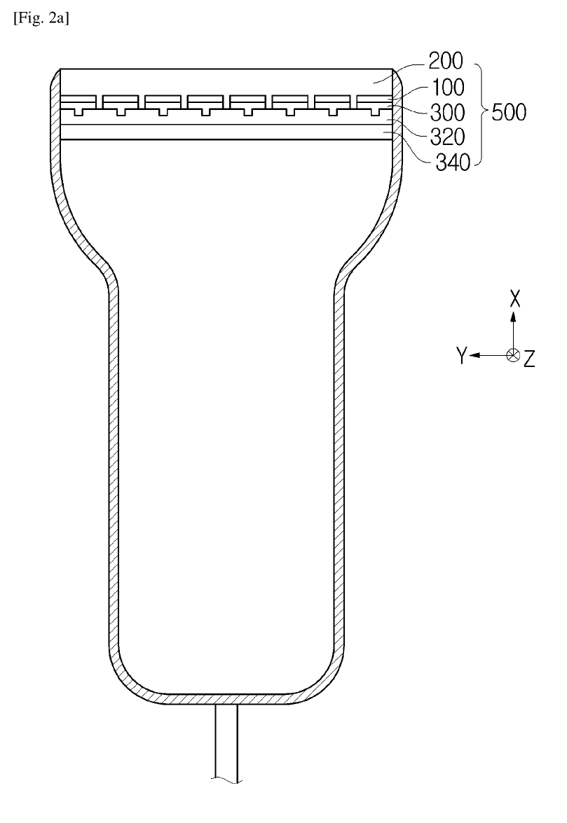

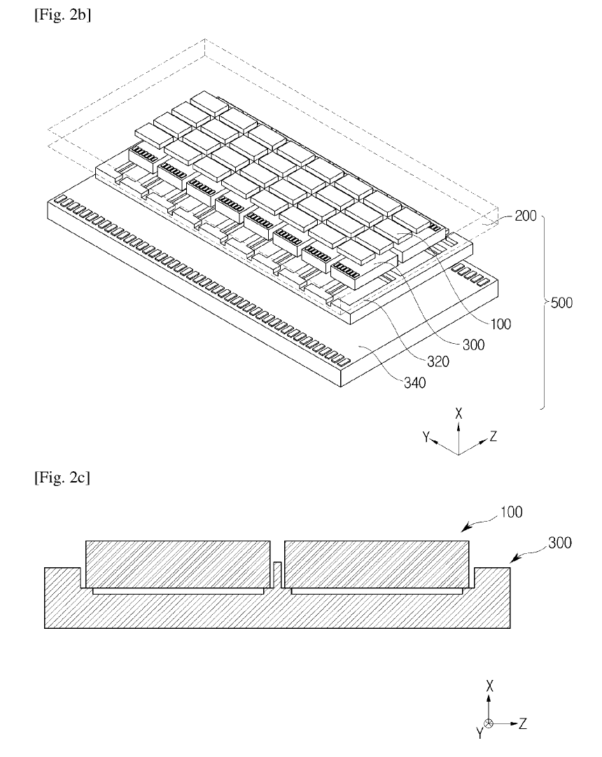

The probe 10 may include one or more transducers 100. The probe 10 may transmit ultrasonic signals to an object, and receive echo ultrasonic waves reflected from the object, using the transducers 100. As illustrated in FIG. 1, the transducers 100 may be arranged in at least one row, and construct a transducer module (500 of FIG. 2A) together with a lens 200 in one end of the probe 10. The object may be a human's or animal's living body, or tissue in a living body, such as vessels, bonds, and muscles, although not limited to these. The transducers 100, the lens 200, and the transducer module 500 will be described in more detail, later.

The main body 30 may accommodate components (for example, a beamforming unit, an image processor, a controller, etc.) of the ultrasound imaging apparatus. The beamforming unit may perform beamforming for compensating for differences between times at which the transducers transmit ultrasonic waves or differences between times at which the transducers receive echo ultrasonic waves. The image processor may acquire an ultrasound image based on the beamformed signals, and perform image conversion, image restoration, and image compression on the ultrasound image.

Also, the controller may generate control signals for controlling the individual components (for example, the probe 10, the beamforming unit, the image processor, etc.) of the ultrasound imaging apparatus. For example, if a user inputs an ultrasonic diagnosis command, the controller may generate a control signal for transmitting ultrasonic waves, and transmit the control signal to the probe 10 or the beamforming unit.

In the main body 30, one or more female connectors (not shown) may be provided. The male connector (not shown) connected to the cable may be physically coupled with one of the female connectors so that the probe 10 can transmit/receive signals to/from the main body 30. For example, a control signal for transmitting ultrasonic waves, generated by the controller, may be transmitted to the probe 100 via the male connector connected to the female connector of the main body 30 and the cable.

In the lower part of the main body 30, a plurality of castors may be provided to fix the ultrasound imaging apparatus at a specific location or to move the ultrasound imaging apparatus in a specific direction.

The input unit 40 allows a user to input commands related to operations of the ultrasonic imaging apparatus. For example, the user may use the input unit 40 to input an ultrasonic diagnosis start command, a command for selecting an area to be diagnosed, a command for selecting a diagnosis type, and a command for selecting a display mode for an ultrasonic image to be finally output. The display mode may be one of an Amplitude mode (A-mode), a Brightness mode (B-mode), a Doppler mode (D-mode), an Elastography mode (E-mode), and a Motion mode (M-mode). A command input through the input unit 40 may be transmitted to the main body 30 through wired/wireless communication.

The input unit 40 may include at least one of a keyboard, a mouse, a trackball, a touch screen, a foot switch, and a foot pedal, although not limited to these.

The input unit 40 may be mounted on the main body 30 as shown in FIG. 1, however, the input unit 40 may be provided below the main body 30 if the input unit 40 is implemented as a foot switch or a foot pedal.

Also, if the input unit 40 is implemented as a Graphical User Interface (GUI) such as a touch screen, that is, if the input unit 40 is softwarily implemented, the input unit 40 may be displayed through the display unit 20 which will be described later.

A probe holder for accommodating the probe 10 may be provided around the input unit 40. A user may put the probe 10 into the probe holder to safely keep the probe 10 when he/she does not use the ultrasound imaging apparatus.

The display unit 20 may display images acquired during ultrasonic diagnosis. The display unit 20 may display images in correspondence to a mode selected by a user, and if no mode has been selected, the display unit 20 may display images in correspondence to a basic mode (for example, B-mode) set in advance by a user.

The display unit 20 may be coupled with and mounted on the main body 30, as shown in FIG. 1, however, the display unit 20 may be detachably mounted on the main body 30. Although not shown in FIG. 1, a sub display for displaying an application (e.g., menus and guidance needed for ultrasonic diagnosis) related to operations of the ultrasound imaging apparatus may be provided.

The display unit 20 may be implemented as a Cathode Ray Tube (CRT), a Liquid Crystal Display (LCD), or a LED display. However, the display unit 20 is not limited to the above-mentioned display devices.

Hereinafter, the transducers 100, the lens 200, and the transducer module 500 will be described in more detail with reference to FIGS. 2A to 11C.

FIG. 2A shows a section of the probe 10 including the transducer module 500, and FIG. 2B is an exploded perspective view of the transducer module 500.

In one end of the probe 10, the transducer module 500 including one or more transducers 100 may be provided, wherein the transducers 100 may be implemented as cMUTs. More specifically, the transducer module 500 may have a structure in which a Printed Circuit Board (PCB) 340, a frame 320, one or more integrated circuits 300, cMUTs 100, and a lens 200 are stacked in this order in a direction in which ultrasonic waves are irradiated, that is, in the increasing direction of the x-axis. In the following description, the direction in which ultrasonic waves are irradiated will be also referred to as “forward” or a “front surface”, and the opposite direction of the direction in which ultrasonic waves are irradiated will be also referred to as “backward” or a “back surface”.

Each transducer 100 may convert an electrical signal applied from a power source into mechanical vibration energy to generate ultrasonic waves, and convert vibration received from an object into an electrical signal. More specifically, if the probe 10 receives current from a power source, such as an external power supply or an internal power storage unit (for example, a battery), the transducers 100 may vibrate according to the received current to generate ultrasonic waves, and irradiate the ultrasonic waves to an object. Also, each transducer 100 may receive echo ultrasonic waves reflected from the object, and vibrate according to the received echo ultrasonic waves to generate current of a frequency corresponding to the vibration frequency.

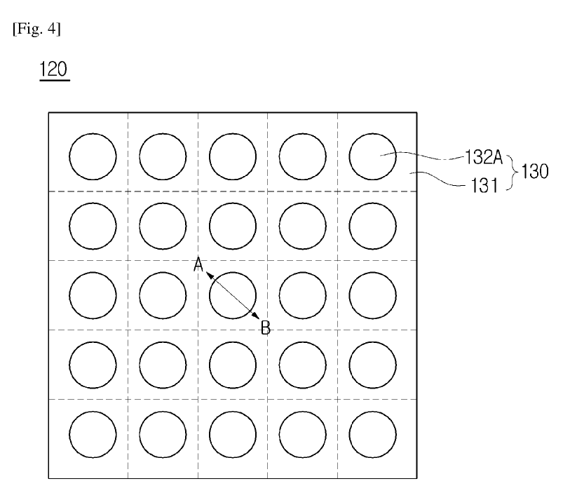

If the transducers 100 are cMUTs as shown in FIGS. 2A and 2B, the transducers 100 may transmit and receive ultrasonic waves using vibration of several hundreds or thousands of micromachined thin films. The cMUT 100 is fabricated based on Micro Electro Mechanical Systems (MEMS) technology. The cMUT 100 may include an anchor (131 of FIG. 5A) such as a wafer, and a membrane (132A of FIG. 5A) to form an air gap (133 of FIG. 5A) together with the anchor 131, wherein the air gap 133 and the membrane 132A may be formed with a thickness of several thousands of A. The anchor 131 and the membrane 132A may form a capacitor with the air gap 133 in between, and if current is applied to the capacitor, the membrane 132A may vibrate to thus generate ultrasonic waves. On the contrary, if the membrane 132A vibrates by echo ultrasonic waves, the capacitance of the capacitor may change, and the change in capacitance may be detected to receive ultrasonic waves. The structure of the cMUT 100 will be described in more detail, later.

The integrated circuit 300 may apply an electrical signal to the cMUT 100 to drive the cMUT 100 to generate ultrasonic waves, and detect an electrical signal output from the cMUT 100 due to echo ultrasonic waves. The integrated circuit 300 may be an Application Specific Integrated Circuit (ASIC).

As shown in FIGS. 2A and 2B, at least one cMUT 100 may be mounted on the front surface of the integrated circuit 300, and electrically connected to the integrated circuit 300 through chip bonding such as flip-chip bonding. More specifically, 1:n flip-chip bonding may be performed in such a manner to fabricate an integrated circuit 300 for fixedly mounting at least one cMUT 100 to be subject to flip-chip bonding, to rest the cMUT 100 on the integrated circuit 300, and then to perform flip-chip bonding. In the “1:n”, 1 represents the number of the integrated circuit 300 subject to flip-chip bonding, and n represents the number of the cMUT(s) 100 subject to flip-chip bonding.

FIG. 2C is a cross-sectional view showing cMUTs mounted on an integrated circuit.

In FIG. 2C, an example in which an integrated circuit 300 includes two accommodating grooves spaced each other, and in each accommodating groove, a cMUT 100 is rested is shown. However, the present disclosure is not limited to this example. According to other examples, one or three cMUTs may be rested in each integrated circuit, or an integrated circuit without any accommodating groove may be used.

Meanwhile, the beamforming unit described above with reference to FIG. 1 may be included in the integrated circuit 300, instead of the main body 30. That is, the integrated circuit 300 may compensate for differences between times at which ultrasonic waves are transmitted to an object or differences between times at which echo ultrasonic waves are received from the object.

The frame 320, which is a tiling frame, may arrange the integrated circuits 300 in the form of tiles. More specifically, as shown in FIG. 2B, in the front surface of the frame 320, one or more accommodating grooves corresponding to the shape, size, and number of the integrated circuits 300 may be formed at predetermined intervals. The integrated circuits 300 may be inserted into the accommodating grooves so that the integrated circuits 300 and the cMUTs 100 mounted on the integrated circuits 300 are arranged in the form of tiles, and the cMUTs 100 are arranged in a 2Dimensional (2D) array.

The frame 320 may be fabricated in various shapes, and the shape of the frame 320 may decide the shape of the transducer module 500 and a direction in which ultrasonic waves are generated. For example, the frame 320 may be fabricated as a planer structure, as shown in FIGS. 2A and 2B, and according to the planar structure of the frame 320, the integrated circuits 300 and the cMUTs 100 may be stacked two-dimensionally so that the cMUTs 100 arranged two-dimensionally can generate ultrasonic waves in the same direction. As another example, the frame 320 may be fabricated as a curved structure, unlike the examples shown in FIGS. 2A and 2B. In this case, the integrated circuits 300 and the cMUTs 100 may also be stacked in a curved shape so that ultrasonic waves can be generated in various directions.

Meanwhile, the frame 320 may be omitted according to embodiments. In this case, the integrated circuits 300 may be directly connected to the PCB 340.

The front surface of the PCB 340 may be directly connected to the integrated circuits 300 through wire bonding or the like, or indirectly connected to the integrated circuits 300 with the frame 320 in between, so that the PCB 340 can transmit an electrical signal received from the main body 30 to the integrated circuits 300 or an electrical signal received from the integrated circuits 300 to the main body 30.

Although not shown in the drawings, in the back surface of the PCB 340, a heat spreader may be further provided to absorb heat generated from the integrated circuits 300. However, since the cMUTs 100 scarcely transmit vibrations due to air gaps described above, a backing for improving residual vibration characteristics does not need to be provided in the back surface of the PCB 340, and accordingly, the structure and manufacturing process of the transducer module 500 can be simplified.

The lens 200 may be coupled with the front surfaces of the cMUTs 100 to protect the cMUTs 100. Also, the lens 200 may enable ultrasonic waves generated from the cMUTs 100 to be emitted from the probe 10 and irradiated to an object, and echo ultrasonic waves reflected from the object to be focused and received by the cMUTs 100. The lens 200 may be Room Temperature Vulcanization (RTV).

In other words, in order to protect the cMUTs 100, the peeling phenomenon of the lens 200 coupled with the front surfaces of the cMUTs 100 may need to be prevented, and in order to improve the transmitting and receiving sensitivity of ultrasonic waves by the cMUTs 100, the interfacial strength between the cMUTs 100 and the lens 200 may need to be improved. Hereinafter, the structure and manufacturing process of the cMUTs 100 for improving the interfacial strength between the cMUTs 100 and the lens 200 will be described in detail.

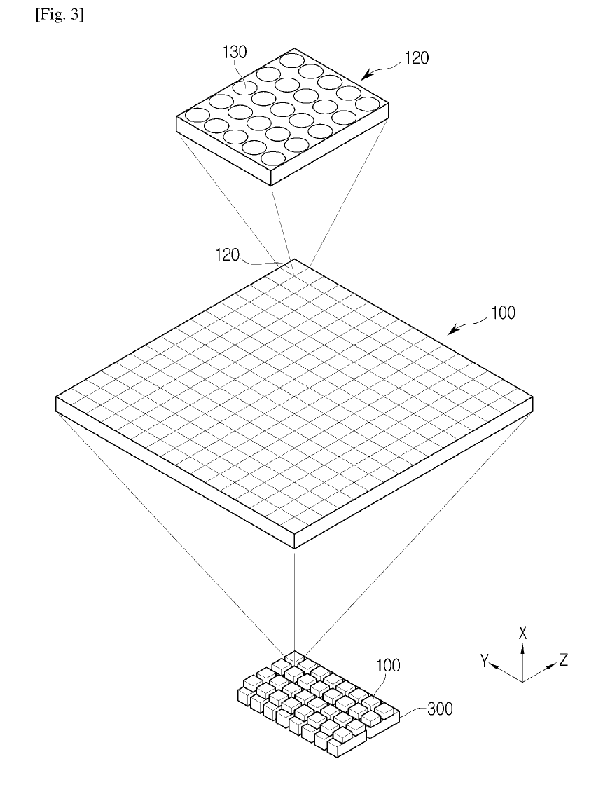

FIG. 3 is a view for describing a configuration of the cMUT 100.

The cMUTs 100 may be arranged in a two-dimensional array with a planar structure, as described above. Each cMUT 100 may be composed of a plurality of elements 120 arranged two-dimensionally, and each element 120 may be composed of a plurality of cells 130 arranged two-dimensionally. Each cell 130 may be composed of an anchor 131, a membrane 132A, and an air gap 133.

For example, as shown in FIG. 3, a transducer module 500 may be composed of 32 cMUTs 100 forming a two-dimensional array of 4×8 size, and each cMUT 100 may be composed of 256 elements 120 forming a two-dimensional array of 16×16 size, so that the cMUTs 100 may be composed of 8192 elements 120.

Also, each element 120 may be composed of 25 cells forming a two-dimensional array of 5×5 size. Accordingly, the cMUTs 100 may be composed of 204,800 cells. In other words, the cMUTs 100 may be composed of 204,800 membranes 132A

FIG. 4 is a plan view of the element 120 shown in FIG. 3, FIG. 5A is a cross-sectional view showing a structure of the cell 130 according to an embodiment of the present disclosure, and FIG. 5B is a cross-sectional view showing a structure of the cell 130 according to another embodiment of the present disclosure.

As shown in FIG. 4, the element 120 composed of 25 cells 130 may have horizontal and vertical sizes of several tens to hundreds of μm, and each cell 130 may have a structure in which a membrane 132A is formed on an anchor 131. FIGS. 5A and 5B show various embodiments of a section of the cell 130 cut along a line A-B of FIG. 4.

More specifically, the upper part of FIG. 5A shows a basic structure of the cell 130, wherein the cell 130 may be formed by forming a first electrode 132B made of a metal such as Al or Au on the anchor 131 made of Si, SiO₂, or SiNₓ, forming a vacuum air gap 133 in front of the first electrode 132B, and then forming a second electrode 132A made of a metal such as Al or Au over the air gap 133. The second electrode 132A may become a membrane, and if current is applied using the first electrode 132B and the membrane 132A as both poles with the air gap 133 in between, the membrane 132A may vibrate to generate ultrasonic waves.

Meanwhile, a sectional structure of the cell 130 for improving the interfacial strength with the lens 200 is shown in the middle part of FIG. 5A. More specifically, at least one groove H1 may be formed in the front surface of the anchor 131 such that the front surface of the anchor 131 forms an uneven structure. The groove H1 is referred to as an anchor groove. If the lens 200 is applied on the anchor 131 having the uneven structure, the lens 200 may fill the anchor groove H1, as shown in the lower part of FIG. 5A. In other words, the inner surfaces of the anchor groove H1 may contact a part of the lens 200 so that a contact area with the lens 200 can be widened. Accordingly, the interfacial strength can be improved regardless of the kind of the metal constituting the membrane 132A.

The upper part of FIG. 5B shows a basic structure of the cell 130, as described above with reference to FIG. 5A, and a sectional structure of the cell 130 according to another embodiment for improving the interfacial strength is shown in FIG. 5B. That is, at least one groove H2 may be formed in the membrane 132A, as well as in the anchor 131 so that the front surfaces of the anchor 131 and the membrane 132A have uneven structures. The at least one groove H1 formed in the anchor 131 will be referred to as an anchor groove, and the at least one groove H2 formed in the membrane 132A will be referred to as a membrane groove, in correspondence to the anchor groove H1.

If the lens 200 is applied on the anchor 131 and the membrane 132A having the uneven structures, the lens 200 may fill both the anchor groove H1 and the membrane groove H2, as shown in the lower part of FIG. 5B. That is, the inner surfaces of the anchor groove H1 and the membrane groove H2 may contact a part of the lens 200 so that a contact area with the lens 200 can be widened, resulting in improvement of interfacial strength. The cell 130 of FIG. 5B in which the front surface of the membrane 132A also has an uneven structure may have greater interfacial strength than the cell 130 of FIG. 5A.

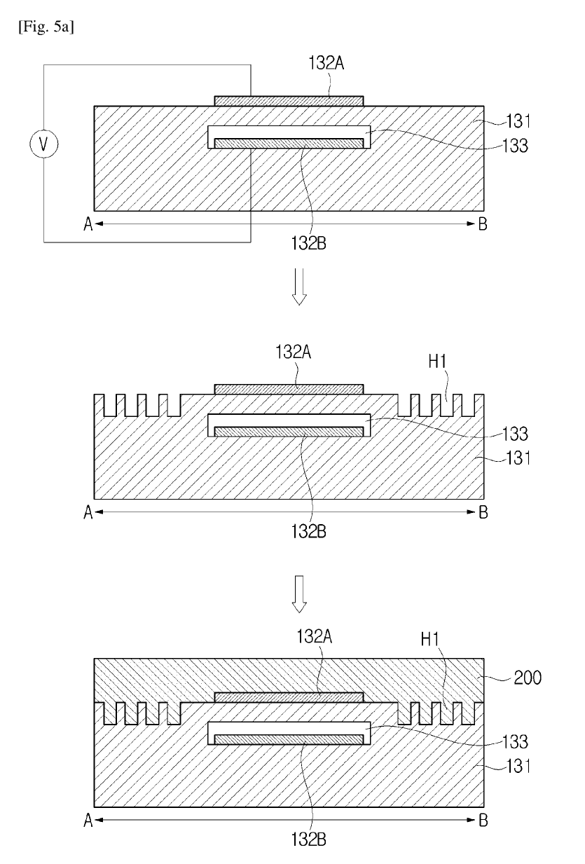

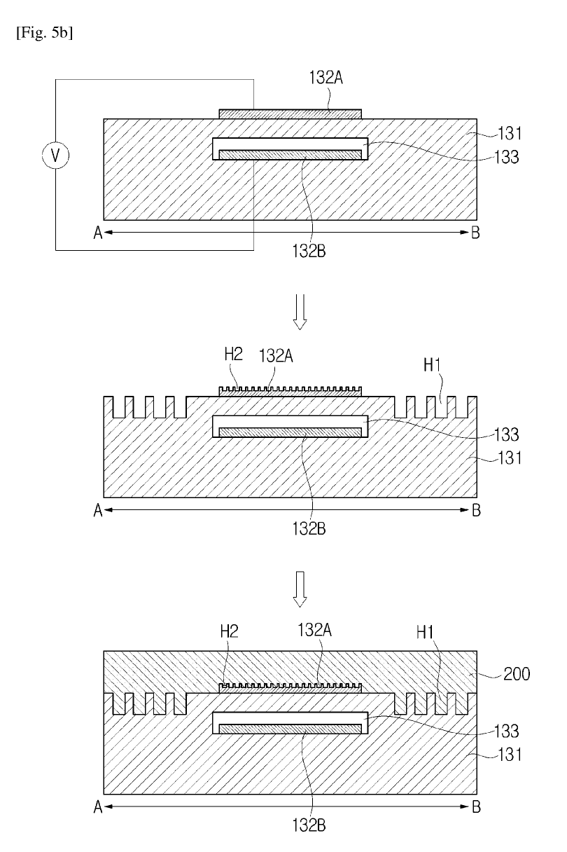

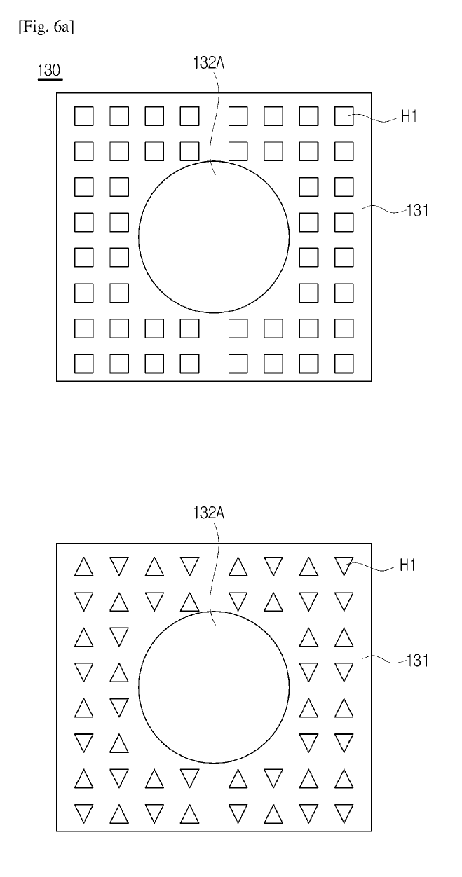

FIG. 6A is a plan view of the cell 130 according to an embodiment of the present disclosure, and FIG. 6B is a plan view of the cell 130 according to another embodiment of the present disclosure.

According to an embodiment, the anchor groove H1 formed in the anchor 131 may have a quadrangular pattern as shown in the upper part of FIG. 6A, or may have a triangular pattern as shown in the lower part of FIG. 6A. However, the anchor groove H1 may have any other pattern.

According to an embodiment, the anchor groove H1 and the membrane groove H2 formed in the anchor 131 and the membrane 132A may have patterns as shown in FIG. 6B. More specifically, the anchor groove H1 and the membrane groove H2 may have the same quadrangular pattern, as shown in the upper part of FIG. 6B, or the anchor groove H1 may have a triangular pattern and the membrane groove H2 may have a quadrangular pattern, as shown in the lower part of FIG. 6B.

That is, the anchor groove H1 and the membrane groove H2 may have the same pattern or different patterns. Also, although the anchor groove H1 and the membrane groove H2 have the same pattern, the size of the anchor groove H1 may be different from that of the membrane groove H2. Also, unlike the examples shown in FIG. 6B, the anchor groove H1 and the membrane groove H2 may have other patterns, instead of the quadrangular or triangular pattern.

Meanwhile, the planar pattern of the anchor groove H1 or the membrane groove H2 will be referred to as a plane pattern.

A method of forming the anchor groove H1 or the membrane groove H2 may be etching, deposition, or bonding. The sectional pattern of the anchor groove H1 or the membrane groove H2 formed by the method may have various shapes. The sectional pattern of the anchor groove H1 or the membrane groove H2 will be simply referred to as a section pattern.

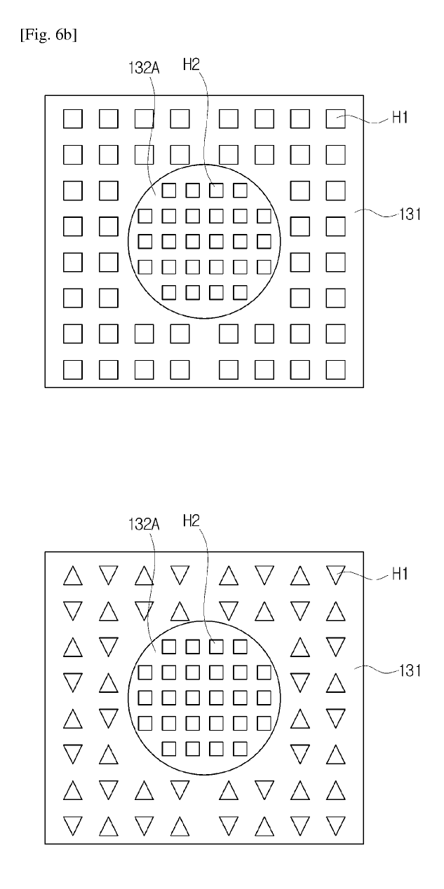

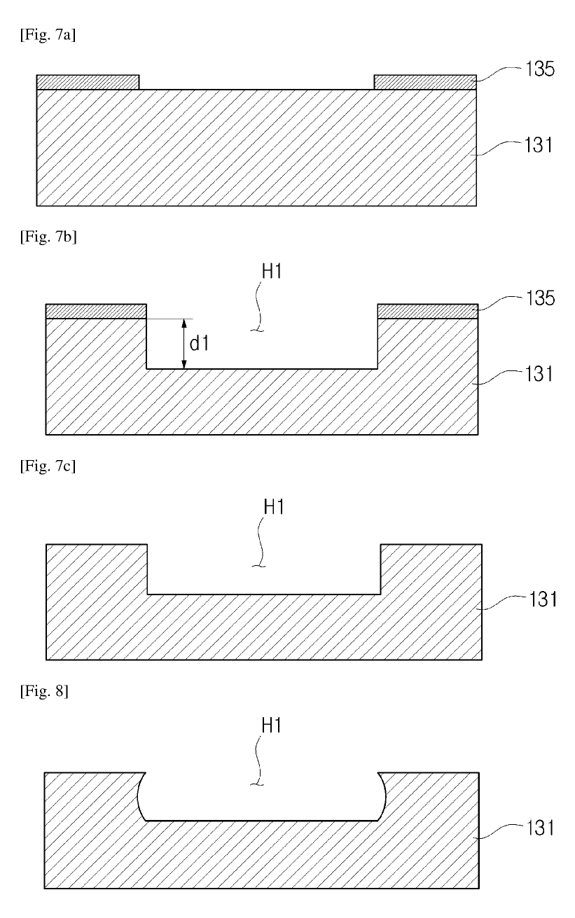

Hereinafter, a method of forming the anchor groove H1, and a section pattern of the anchor groove H1 will be described in detail with reference to FIGS. 7A to 11C. Also, a method of forming the membrane groove H2, and a section pattern of the membrane groove H2 will be described based on differences from the anchor groove H1.

The anchor groove H1 may be formed by etching the anchor 131 through dry etching or wet etching.

More specifically, in order to form the anchor groove H1 at a depth of several μm, dry etching using fluorine-based gas, such as CF₄ or SF₆, or chlorine-based gas such as CCl₄ may be applied, or wet etching using Buffered Oxide Etchant (BOE) may be applied. Also, in order to form the anchor groove H1 at a depth of several tens or hundreds of μm, dry etching based on Deep Reactive-Ion Etching (DRIE) may be applied, or wet etching using KOH or TMAH may be applied.

FIGS. 7A to 7C are views for describing an example of an etching process.

As shown in FIG. 7A, photo resist may be applied on the front surface of the anchor 131 to form a mask layer 135. A process of forming the mask layer 135 will be described in detail, below.

Photo resist may be applied on the entire front surface of the anchor 131, and light such as ultraviolet light may be selectively irradiated on the surface of the photo resist. Thereafter, a developer may be used to remove the photo resist at an area on which the light has been irradiated. For example, if the photo resist is positive resist, the photo resist at the area on which the light has been irradiated may be resolved or softened by the light and then removed through the developer, and photo resist at the remaining area on which no light has been irradiated may be hardened.

The hardened photo resist may form the mask layer 135. The area of the photo resist removed through the developer may form a plane pattern of the anchor groove H1. In other words, light may be selectively irradiated based on a plane pattern of the anchor groove H1 that is to be formed.

Then, etching may be performed using the mask layer 135 as an etching prevention film. At this time, dry etching using fluorine-based gas may be applied. That is, CF₄ or SF₆ in the state of plasma may be applied on the anchor 131 and the mask layer 135 to form the anchor groove H1. The formed anchor groove H1 may have a section pattern of a rectangular shape whose one side opens at an area on which no mask layer is formed, as shown in FIG. 7B.

After the anchor groove H1 is formed, the mask layer 135 may be removed, as shown in FIG. 7C.

As described above, according to the dry etching using fluorine-based gas, the anchor groove H1 having the section pattern of the rectangular shape may be formed, wherein the depth d1 of the anchor groove H1 is several μm or less.

However, wet etching may be applied so that the section pattern of the anchor groove H1 has another shape. In other words, when etching is performed using the mask layer 135 as an etching prevention film, an etchant instead of plasma may be used.

FIG. 8 is a cross-sectional view showing an example of the anchor 131 etched by an etchant, wherein the etchant may be BOE.

The anchor groove H1 may be formed by immersing the anchor 131 on which the mask layer 135 has been formed in BOD which is an etchant, and the formed anchor groove H1 may have a section pattern with curved sides. The depth of the anchor groove H1 may be several μm or less, like when fluorine-based gas is used.

FIG. 9 is a cross-sectional view showing another example of the anchor 131 etched by an etchant, wherein the etchant may be a KOH or TMAH solution.

That is, the anchor groove H1 may be formed by immersing the anchor 131 on which the mask layer 135 has been formed in KOH or TMAH which is an etchant. The formed anchor groove H1 may have a section pattern of a trapezoid shape which has a gradient of 0° and whose one side opens, as shown in FIG. 9. The depth of the anchor groove H1 may be several tens or hundreds of μm, unlike when fluorine-based gas or BOD is used.

In order to form the anchor groove H1 at a depth of several tens or hundreds of μm, dry etching based on DRIE may be used.

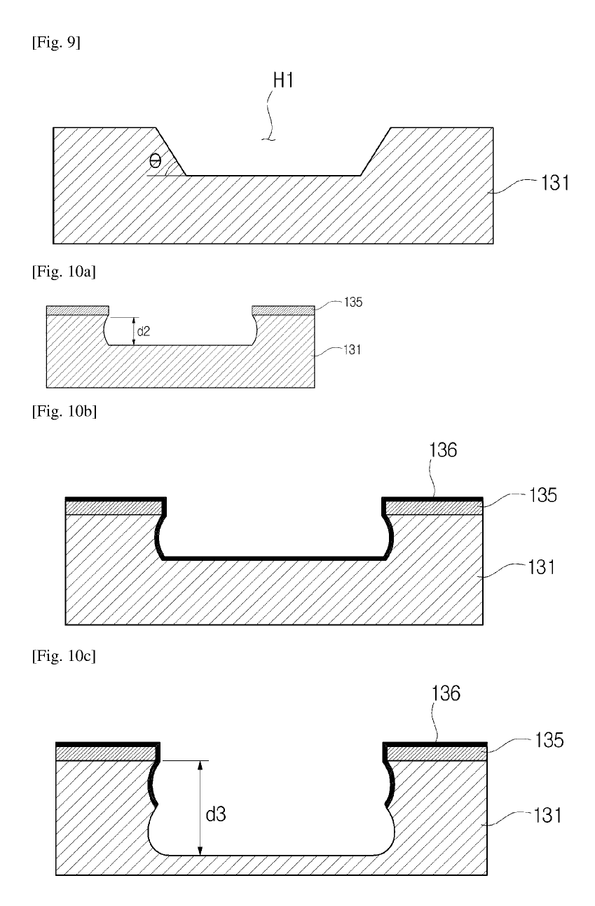

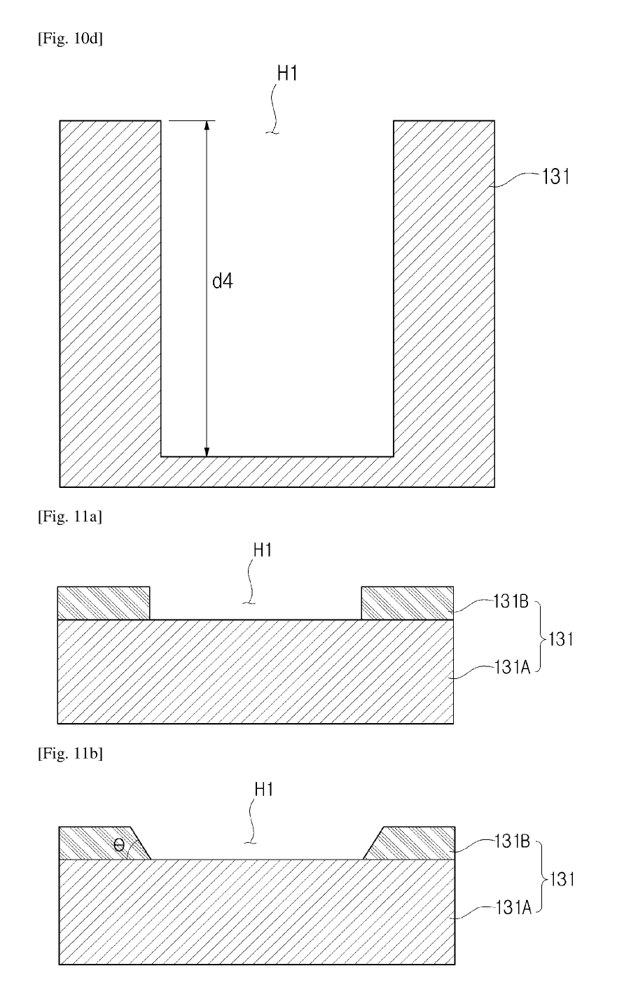

FIGS. 10A to 10D are views for describing an etching process according to DRIE.

Photo resist may be applied on the front surface of the anchor 131 to form a first mask layer 135. A process of forming the first mask layer 135 is the same as the process of forming the mask layer 135 as described above with reference to FIG. 7A, and accordingly, a detailed description thereof will be omitted.

Then, first etching may be performed using the first mask layer 135 as an etching prevention film. More specifically, SF₆ in the state of plasma may be applied on the anchor 131 and the first mask layer 135. Ions SF₅⁺ constituting SF₆ may react with the anchor 131 at an area on which no first mask layer 135 has been formed, so as to form a first groove at a depth of d2, as shown in FIG. 10A.

Then, polymers, such as C₄F₈ or CHF₃, may be applied on the front surfaces of the first mask layer 135 and the anchor 131 in which the first groove has been formed, so as to form a second mask layer 136, as shown in FIG. 10B.

Then, SF₆ in the state of plasma may be applied on the second mask layer 136. Ions SF₅⁺ may protect sidewalls and react with the anchor 131 to thus perform second etching, as shown in FIG. 10C. Accordingly, a second groove may be formed at a depth of d3. That is, due to the second etching, a second groove having a depth of d3 that is deeper than the depth d2 of the first groove may be formed.

Thereafter, a process of forming the second mask layer 136 and performing the second etching may be repeatedly performed, and then the first mask layer 135 may be removed. As a result, an anchor groove H1 having a depth of d4 may be formed, as shown in FIG. 10D, wherein the depth d4 may be several tens or hundreds of μm.

The membrane groove H2 may also be formed using etching, like the anchor groove H1, however, the membrane groove H2 may have limitation in depth.

More specifically, since the cMUT 100 generates ultrasonic waves and receives echo ultrasonic waves using the vibration of the membrane 132A, the transmitting and receiving efficiency of ultrasonic waves may be influenced by the resonance frequency of the membrane 132A. For example, the transmittance of ultrasonic waves may vary depending on the resonance frequency of the membrane 132A. The resonance frequency may be influenced by the mass of the membrane 132A, as seen in Equation (1).

$${f}_{c} ∝ \frac{1}{m} , \qquad ( 1 )$$

where f_{c} represents the resonance frequency of the membrane 132A, and m represents the mass of the membrane 132A.

That is, since the resonance frequency of the membrane 132A is in inverse proportion to the mass of the membrane 132A, if the mass of the membrane 132A is excessively reduced by etching, the resonance frequency of the membrane 132A may increase excessively. As a result, the transmittance of ultrasonic waves may deteriorate.

Accordingly, there is a method of etching the anchor 131 or the membrane 132A at the deeper depth in order to widen a contact area with the lens 200. However, the etching depth of the membrane groove H2 is limited. For example, if the thickness of the membrane 132A is 0.2 μm, the depth of the membrane groove H2 may not exceed 0.02 μm.

As such, the membrane groove H2 needs to have a depth of several μm, and accordingly, dry etching of providing chlorine-based gas such as CCl₄ in the state of plasma or wet etching of immersing a target in an etchant for metal may be applied to etch the membrane 132A.

The anchor groove H1 may be formed through deposition.

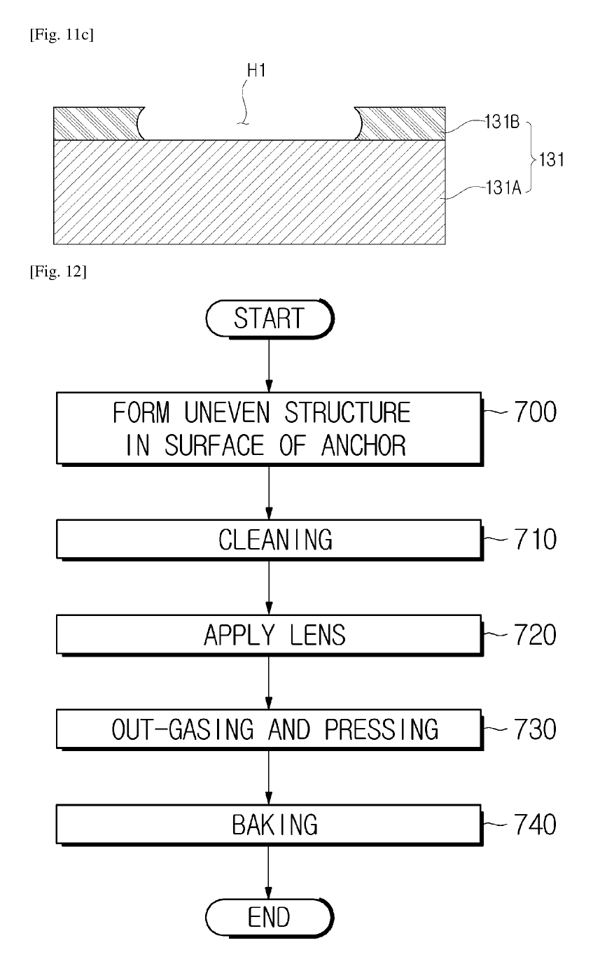

More specifically, polysilicon, SiNₓ, or SiO₂ may be deposited on a layer 131A formed with Si, SiO₂, or SiNₓ to form a deposited film 131B as shown in FIGS. 11A to 11C. By forming the deposited film 131B, an anchor 131 with an anchor groove H1 may be formed, wherein a section pattern of the anchor groove H1 may be a pattern with vertical sidewalls, inclined sidewalls, or curved sidewalls, as respectively shown in FIGS. 11A, 11B, and 11C.

A membrane groove H2 may also be formed using a method of depositing polysilicon, SiNₓ, or SiO₂. More specifically, a deposited film may be formed with polysilicon, SiNₓ, or SiO₂ on a metal layer formed with Al or Au to form a membrane groove H2 with vertical sidewalls, inclined sidewalls, or curved sidewalls, so that the membrane 132A may be formed with the metal layer and the deposited film.

At this time, the deposited film may be formed in consideration of the deformation temperature or melting temperature of the metal layer. For example, if the metal layer is formed with Al, the deposition film may be deposited using Plasma-Enhanced Chemical Vapor Deposition (PE-CVD), sputtering, or an evaporator, which allows deposition at a lower temperature than 450° C. that is a melting point of Al.

The anchor groove H1 or the membrane groove H2 may be formed through bonding, such as eutectic bonding, silicon direct bonding, or epoxy bonding.

The eutectic bonding is based on metal binding allowing an eutectic process, such as Sn—Au and Sn—Ag, the silicon direct bonding is performed by forming SiO₂ between Si and Si and then applying an appropriate temperature and pressure to the SiO₂, and epoxy bonding is performed by applying epoxy. The eutectic bonding, the silicon direct bonding, and the epoxy bonding have been well-known in the art, and accordingly, detailed descriptions thereof will be omitted.

Meanwhile, the depth of the membrane groove H2 that is formed through deposition or bonding may also be limited.

The configuration of the transducer module 500, more specifically, the anchor 131 and the membrane 132a having uneven structures for improving the interfacial strength with the lens 200 have been described above. That is, the anchor 131 in which the anchor groove H1 has been formed and the membrane 132A in which the membrane groove H2 has been formed have been described above. Hereinafter, a method of manufacturing the transducer module 500 including the anchor groove H1 or the membrane groove H2 will be described in detail with reference to a flowchart.

FIG. 12 is a flowchart illustrating a method of manufacturing a probe, according to an embodiment of the present disclosure.

Referring to FIG. 12, in order for the cMUT 100 to have an uneven structure, at least one anchor groove H1 may be formed in the front surface of the cMUT 100, more specifically, in the front surface of the anchor 131, in operation 700.

A method of forming the anchor groove H1 may be etching, deposition, or bonding.

In the case of etching, in order to form the anchor groove H1 at a depth of several μm, dry etching using fluorine-based gas, such as CF₄ or SF₆, or chlorine-based gas such as CCl₄ may be applied, or wet etching using BOE may be applied. Also, in order to form the anchor groove H1 at a depth of several tens or hundreds of μm, dry etching based on DRIE may be applied, or wet etching using KOH or TMAH may be applied.

In the case of deposition, the anchor groove H1 may be formed by forming a deposited film with polysilicon, SiNₓ, or SiO₂.

In the case of bonding, the anchor groove H1 may be formed using at least one of eutectic bonding, silicon direct bonding, and epoxy bonding.

The anchor grooves H1 formed through etching, deposition, or bonding may have a repeated pattern in which polygons, such as quadrangles or triangles, are arranged at regular intervals, as shown in FIG. 6A. Also, a section pattern of the anchor grooves H1 may be a repeated pattern with vertical sidewalls, inclined sidewalls, or curved sidewalls, as shown in FIGS. 11A, 11B, and 11C.

Then, the cMUT 100 may be cleaned, in operation 710. In other words, the cMUT 100 may be cleaned by isopropyl alcohol or isopropanol (IPA) or ultrasonic waves in a cleaning hood.

Then, the lens 200 may be applied on the front surface of the cMUT 100 in which the uneven structure has been formed, in operation 720. Herein, the lens 200 may be RTV.

Successively, out-gasing and pressing may be performed, in operation 730. For example, the lens 200 may be out-gassed at 3×10⁻³ Torr for 15 minutes in a vacuum chamber, and then, pressed onto the cMUT 100 by the power of a spring.

Then, baking may be performed in an oven so that the lens 200 pressed on the cMUT 100 may be hardened, in operation 740. At this time, the baking may be performed at 50° C. for 6 hours.

According to the manufacturing method, the inner surfaces of the anchor groove H1 may contact a part of the lens 200 so that a contact area with the lens 200 can be widened. Accordingly, the interfacial strength between the cMUT 100 and the lens 200 can be improved regardless of the kind of the metal constituting the membrane 132A.

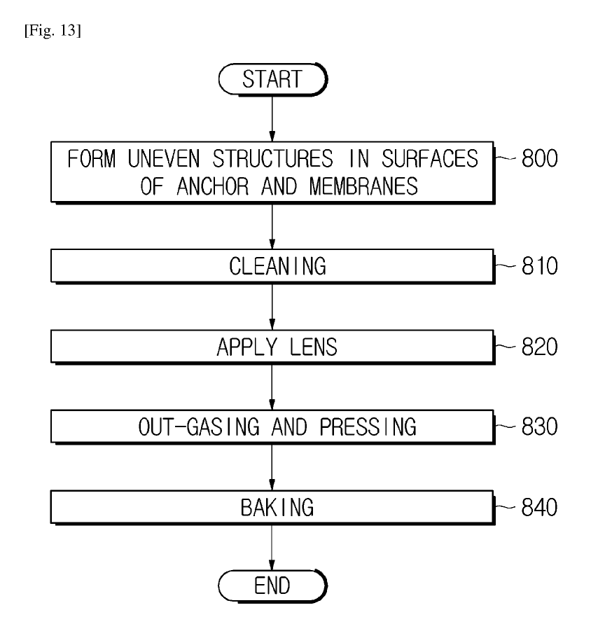

FIG. 13 is a flowchart illustrating a method of manufacturing a probe, according to another embodiment of the present disclosure.

Referring to FIG. 13, in order for the cMUT 100 to have an uneven structure, at least one anchor groove H1 may be formed in the front surface of the cMUT 100, more specifically, in the front surface of the anchor 131, and at least one membrane groove H2 may be formed in the front surface of the membrane 132A, in operation 800.

A method of forming the anchor groove H1 and the membrane groove H2 may be etching, deposition, or bonding. A method of forming the anchor groove H1 may be the same method as described above with reference to FIG. 12, and accordingly, hereinafter, a method of forming the membrane groove H2 will be described.

In the case of etching, dry etching of providing chlorine-based gas such as CCl₄ in the state of plasma, or wet etching of immersing a target in an etchant for metal may be applied.

In the case of deposition, the membrane groove H2 may be formed by forming a deposited film with polysilicon, SiNₓ, or SiO₂ on a metal layer formed with Al or Au. At this time, the deposition film may be deposited using PE-CVD, sputtering, or an evaporator, which allows deposition at a lower temperature than a melting point of the metal layer.

In the case of bonding, the membrane groove H2 may be formed using at least one of eutectic bonding, silicon direct bonding, and epoxy bonding.

The anchor grooves H1 and the membrane grooves H2 formed through etching, deposition, or bonding may have a repeated pattern in which polygons, such as quadrangles or triangles, are arranged at regular intervals, as shown in FIG. 6A. Also, the anchor grooves H1 and the membrane grooves H2 may have a circular, repeated pattern. Also, a section pattern of the anchor grooves H1 and the membrane grooves H2 may be a repeated pattern with vertical sidewalls, inclined sidewalls, or curved sidewalls, as shown in FIGS. 11A, 11B, and 11C.

The anchor grooves H1 and the membrane grooves H2 may have the same pattern or different patterns. However, although the anchor grooves H1 and the membrane grooves H2 have the same pattern, the sizes of the anchor grooves H1 may be different from those of the membrane grooves H2.

Meanwhile, in order to widen a contact area with the lens 200, the depths of the anchor groove H1 and the membrane groove H2 may increase. However, the depth of the membrane groove H2 may be limited according to the transmitting and receiving efficiency of ultrasonic waves, and correlation between the resonance frequency and mass. For example, if the thickness of the membrane 132A is 0.2 μm, the depth of the membrane groove H2 may not exceed 0.02 μm.

Then, operation 810 of cleaning the cMUT 100, operation 820 of applying a lens 200, out-gasing and pressing operation 830, and baking operation 840 may be performed sequentially. The operations 810 to 840 may be the same as the operations 710 to 740 described above with reference to FIG. 12, and accordingly, detailed descriptions thereof will be omitted.

According to the manufacturing method, the inner surfaces of the anchor groove H1 and the membrane groove H2 may contact a part of the lens 200 so that a contact area with the lens 200 can be widened. Accordingly, the interfacial strength between the cMUT 100 and the lens 200 can be improved regardless of the kind of the metal constituting the membrane 132A.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

1. A Capacitive Micromachined Ultrasonic Transducer (cMUT) comprising:
an anchor; and
a membrane coupled with the anchor,
wherein the anchor comprises at least one anchor groove.

2. The cMUT according to claim 1, wherein the membrane comprises at least one membrane groove.

3. The cMUT according to claim 1, wherein the anchor is formed with at least one of Si, SiO₂, and SiNₓ.

4. The cMUT according to claim 2, wherein the membrane is formed with at least one of Al and Au.

5. The cMUT according to claim 2, wherein an opened side of the anchor groove or an opened side of the membrane groove has a polygon pattern or a circular pattern.

6. The cMUT according to claim 2, wherein an opened side of the anchor groove and an opened side of the membrane have different shapes of patterns.

7. The cMUT according to claim 2, wherein an opened side of the anchor groove and an opened side of the membrane have different sizes of patterns.

8. The cMUT according to claim 2, wherein sidewalls of the anchor groove or sidewalls of the membrane groove are curved.

9. The cMUT according to claim 2, wherein sidewalls of the anchor groove or sidewalls of the membrane groove are inclined.

10. The cMUT according to claim 2, wherein the anchor groove and the membrane groove are formed at different depths.

11. (canceled)

12. A probe comprising:
a Capacitive Micromachined Ultrasonic Transducer (cMUT) comprising an anchor, and a membrane coupled with the anchor; and
a lens coupled with the cMUT,
wherein the anchor comprises a least one anchor groove.

13. The probe according to claim 12, wherein the membrane comprises at least one membrane groove.

14. The probe according to claim 13, wherein an opened side of the anchor groove or an opened side of the membrane groove has a polygon pattern or a circular pattern.

15. The probe according to claim 13, wherein an opened side of the anchor groove and an opened side of the membrane have different shapes of patterns.

16. The probe according to claim 13, wherein an opened side of the anchor groove and an opened side of the membrane have different sizes of patterns.

17. The probe according to claim 13, wherein sidewalls of the anchor groove or sidewalls of the membrane groove are curved.

18. The cMUT according to claim 13, wherein sidewalls of the anchor groove or sidewalls of the membrane groove are inclined.

19. The cMUT according to claim 13, wherein the anchor groove and the membrane groove are formed at different depths.

20. A method of manufacturing a probe, comprising:
forming at least one anchor groove in an anchor; and
applying a lens on the anchor and a membrane coupled with the anchor.

21. The method according to claim 20, further comprising forming at least one membrane groove in the membrane.

22.-28. (canceled)
